(19) United States
(12) Patent Application Publication
(10) Pub. No.: US 2004/0225361 A1
(43) Pub. Date: Nov. 11, 2004

(54) Intervertebral disk nuclear augmentation system

(76) Inventors: Bradley J. Glenn, Reno, NV (US); Gary A. Schneiderman, Sacramento, CA (US)

Correspondence Address: BRADLEY P. HEISLER, HEISLER & ASSOCIATES, 3017 DOUGLAS BOULEVARD, SUTIE 300, ROSEVILLE, CA, 95661, US

(21) Appl. No.: 10/802,128

(22) Filed: Mar. 15, 2004

Related U.S. Application Data
(60) Provisional application No. 60/454,418, filed on Mar. 14, 2003.

(51) Int. Cl. A61F 2/44

(52) U.S. Cl. 623/17.12; 623/17.16

(57) ABSTRACT

Various implants are provided to at least partially replace a nucleus of a spinal disk. The implants are spring-like in nature. In one embodiment, a helical spring is provided with various different unique outlines to act as the implant. The helical spring is oriented with a center line substantially perpendicular to the spine and to a direction of compression loads experienced within the disk space. The helical spring or other implant is preferably delivered through a delivery cannula which has a size which is smaller than a cross-sectional size of the implant. The implant is preferably formed of nickel titanium or otherwise configured so that it can be compressed significantly within the delivery cannula and then become enlarged after being advanced out of the delivery cannula and into the intervertebral space. In other embodiments the implant is generally cylindrical and expandable in height after delivery.

### CROSS-REFERENCE TO RELATED APPLICATIONS

This application claims benefit under Title 35, United States Code §119(e) of U.S. Provisional Application No. 60/454,418 filed on Mar. 14, 2003.

### FIELD OF THE INVENTION

The following invention relates to implants for surgical placement within an intervertebral space between two adjacent vertebrae to replace a nucleus of the disk and optionally to support the adjacent vertebrae during fusion of the two vertebrae together. More particularly, this invention relates to implants which are in the form of a spring to provide a resilient structure to replace the disk nucleus and function as an artificial disk nucleus.

### BACKGROUND OF THE INVENTION

A healthy human spine includes a series of vertebrae with disks located in an intervertebral space between each of the adjacent vertebrae. Each of the disks includes the annulus fibrosis around a perimeter and the nucleus within a center region. The disk generally functions as a form of shock absorber to absorb typically vertical axial loads experienced by the spine. The annulus holds the adjacent vertebrae securely together while the nucleus has a somewhat resilient character applying force to keep the vertebrae apart, but capable of vertical compression and horizontal expansion to some extent to absorb loads experienced by the spine.

Numerous different spine disorders can cause the disk to cease to function properly. One such condition is referred to as a “herniated” disk where a portion of the disk nucleus escapes through a hole in the surrounding annulus. If the herniated disk puts pressure on nerves adjacent the spine, an unacceptable level of discomfort can result.

Two known treatments to address disk malfunction include spinal fusion and disk nucleus replacement. With spinal fusion, the disk nucleus, and optionally the annulus, are removed. The vertebrae adjacent the space are fixed in position, typically by some structure placed between the two vertebrae. A bone growth medium is placed within this space to encourage the adjacent vertebrae to grow into this space and to grow together. This procedure is not entirely desirable because the space between the vertebrae no longer acts as a shock absorber as the healthy disk does.

With disk nucleus replacement, structures can be provided after the nucleus has been removed which act in a somewhat similar fashion to the disk nucleus. One such disk nucleus replacement device is the intervertebral prosthesis taught by Husson in U.S. Pat. No. 6,610,094. An appropriate length of elongate flexible material is inserted through a small opening in the annulus with the prosthesis allowed to spiral within the interior where the nucleus was removed, until the space within the annulus is filled with the prosthesis. When the space is filled, excess portions of the prosthesis are cut off. A somewhat similar implant is taught by Trieu in U.S. Pat. No. 6,620,196.

While prior art nucleus replacement implants show one system for nuclear replacement, further improvement in the configuration and delivery of such devices would provide a still greater benefit. Particularly, it is desirable that the implant have a predictable and high degree of resiliency, even when cycled through potentially millions of load cycles. Also, it is desirable that such an implant would be delivered into the intervertebral space in as minimally invasive a procedure as possible. Of particular benefit is delivery of the implant through a delivery cannula having a diameter which is less than a final diameter of the implant itself, such that a size of any incisions, and the disruption to the annulus can be minimized.

### SUMMARY OF THE INVENTION

This invention provides an intervertebral space implant preferably for location within the annulus and replacing the nucleus of the disk, or at least a portion thereof, while preferably avoiding the need for spinal fusion, but optionally acting to support adjacent vertebrae should spinal fusion be needed. The implant according to the preferred embodiment is configured as a helical spring. The helical spring includes multiple turns surrounding a center line. The center line can be linear, curved or have other contours. The center line is located between the vertebrae when the implant is located within the intervertebral space, such that the helical spring is in an orientation generally laying on its side. Hence, the spring is not loaded in typical fashion with compression forces pushing the ends toward each other or extension forces drawing the ends away from each other. Rather, the helical spring is loaded laterally. In such an arrangement, a single implant can support a relatively large area while still having a relatively small cross-sectional size for delivery through a particularly small delivery cannula.

The implant can have various different geometry particulars depending on the particular performance desired for the implant. For instance, a center of the implant can have a greater diameter than ends of the implant either to conform to contours of adjacent vertebrae or to provide a variable spring force effect based on the amount of compression load experienced, in a fashion somewhat akin to that of a leaf-spring. Similarly, the implant can have the general form of an ellipsoid so that it is somewhat flattened to maximize support surface in contact with adjacent vertebrae. The implant can also be arced if desired to conform with the geometry of the adjacent vertebrae. The implant can have a larger height at a front end and a smaller height at a rear end so that the implant can provide a greater amount of spacing between the adjacent vertebrae on an anterior side of the space than at a posterior side of the space, where such a positioning of the adjacent vertebrae is considered desirable.

Adjacent turns of the helical spring can be spaced from each other when the spring is at rest or can be directly adjacent each other and abutting each other when the spring is at rest. If the turns are abutting, or sufficiently close to each other, surfaces of the turns can be configured in a mating fashion so that adjacent turns lock together somewhat to allow adjacent turns to support one another when in use. The helical spring could also be replaced with an analogous shell spring having a “C-shaped” cross-section maintained between ends of the shell spring and with a slit along one side of the shell spring to facilitate compression thereof as well as temporary collapse for delivery to the intervertebral space.

While the implant could be delivered into the intervertebral space utilizing direct open surgical procedures or any other delivery methodology, most preferably delivery occurs through a small delivery cannula accessing the intervertebral space either posteriorly or lateral to the intervertebral space. The cannula preferably has a smaller diameter than that of the implant. The spring can be compressed in various different ways. For instance, it can be somewhat unraveled into an elongate gradually spiraling helix with only a few turns, but not exceeding its elastic limit, so that once delivered it takes on its desired final shape. It could alternatively be compressed so that each of the turns has a smaller diameter but with the number of turns actually increasing along with a length of the implant until implantation has occurred.

Most preferably, the implant is formed from a nickel titanium alloy which has “shape memory” characteristics. Particularly, many nickel titanium alloys have a soft martensite phase when dropped below a transition temperature and a hard austenite phase when raised above the transition temperature. By cooling the implant to its martensite phase, it can be easily manipulated as identified above for placement within a delivery cannula. When the implant is later released from the delivery cannula, it is heated up to above the transition temperature and into its austenite phase where it becomes harder and through its shape memory automatically changes its geometry to the larger uncompressed geometry desired.

An analogous implant can use a resilient cylindrical material spaced between two end caps which can be drawn together to cause the resilient material to expand outwardly.

### OBJECTS OF THE INVENTION

Accordingly, a primary object of the present invention is to provide an implant for placement within an intervertebral space within a spine, at least partially replacing a nucleus of the disk in both position and function so that the disk space can continue to function somewhat similarly to its original function.

Another object of the present invention is to treat a damaged spinal disk by implanting a resilient structure within the nucleus of the disk to allow the disk to continue to function effectively.

Another object of the present invention is to provide a spinal disk nuclear augmentation system which utilizes an implant spring which gives the disk similar performance characteristics as a healthy disk.

Another object of the present invention is to provide a nuclear implant which can be readily delivered through a delivery cannula which has a smaller diameter than the implant being delivered.

Another object of the present invention is to provide an implant which can either function similarly to a disk nucleus or fix vertebrae adjacent the intervertebral space sufficiently so that spinal fusion can be performed if needed.

Another object of the present invention is to provide a disk nucleus replacement which can handle the loads and cycles necessary to provide effective replacement for the disk nucleus.

Other further objects of the present invention will become apparent from a careful reading of the included drawing figures, the claims and detailed description of the invention.

### BRIEF DESCRIPTION OF THE DRAWINGS

FIG. 1 is a side elevation view of a spine with an implant according to a first embodiment located within an intervertebral space thereof.

FIG. 2 is a top plan view of that which is shown in FIG. 1.

FIG. 3 is a front full sectional view of that which is shown in FIG. 1.

FIG. 4 is a perspective view of the implant of FIG. 1.

FIG. 5 is a perspective view of the implant of FIG. 1 as it is being advanced out of a delivery cannula and is taking on its curving shape.

FIG. 6 is a side elevation view of a human spine with a second embodiment implant positioned within the intervertebral space between two adjacent vertebrae of the spine.

FIG. 7 is a top plan view of that which is shown in FIG. 6.

FIG. 8 is a full sectional view of that which is shown in FIG. 6.

FIG. 9 is a perspective view of the implant of FIG. 6.

FIGS. 10-13 are top plan views of linear and curving delivery cannulas for delivering implants similar to that which is shown in FIGS. 6-9.

FIG. 14 is a full sectional view of a threaded cannula with the implant therein.

FIGS. 15 and 16 are top plan views revealing stages in delivery of an implant through use of a threaded cannula.

FIG. 17 is an end view of a third embodiment implant of this invention.

FIG. 18 is a front elevation view of that which is shown in FIG. 17.

FIG. 19 is a top plan view of that which is shown in FIG. 18.

FIG. 20 is a top plan view of a fourth embodiment implant of this invention which exhibits a curving contour.

FIG. 21 is a front elevation view of a fifth embodiment implant according to this invention where adjacent turns of the helical spring of the implant are directly adjacent each other when the implant is at rest.

FIG. 22 is a front elevation view of that which is shown in FIG. 21 when ends thereof are pulled away from each other.

FIGS. 23-25 provide details of three separate embodiments of locking surfaces of adjacent turns of the implant of FIG. 21 to facilitate adjacent turns supporting each other.

FIG. 26 is a perspective view of a sixth embodiment implant according to this invention which exhibits a conical taper outline.

FIG. 27 is a top plan view of the implant of FIG. 26 being delivered into position.

FIG. 28 is a side elevation view of the implant of FIG. 26 after implantation is complete.

FIG. 29 is a top plan view of a seventh embodiment implant according to this invention which is in the form of a shell spring.

FIG. 30 is a rear elevation view of that which is shown in FIG. 29.

FIG. 31 is an end view of that which is shown in FIGS. 29 and 30 shown in the form of a slice taken from a mid region of that which is shown in FIGS. 29 and 30.

FIG. 32 is an end view similar to that which is shown in FIG. 31 but with the shell spring compressed such as before delivery.

FIGS. 33-35 are front elevation full sectional views of an eighth embodiment implant according to this invention illustrating three stages in the process of compressing the implant to cause the implant to achieve varying degrees of radial expansion to appropriately fit within the disk space.

### DESCRIPTION OF THE PREFERRED EMBODIMENT

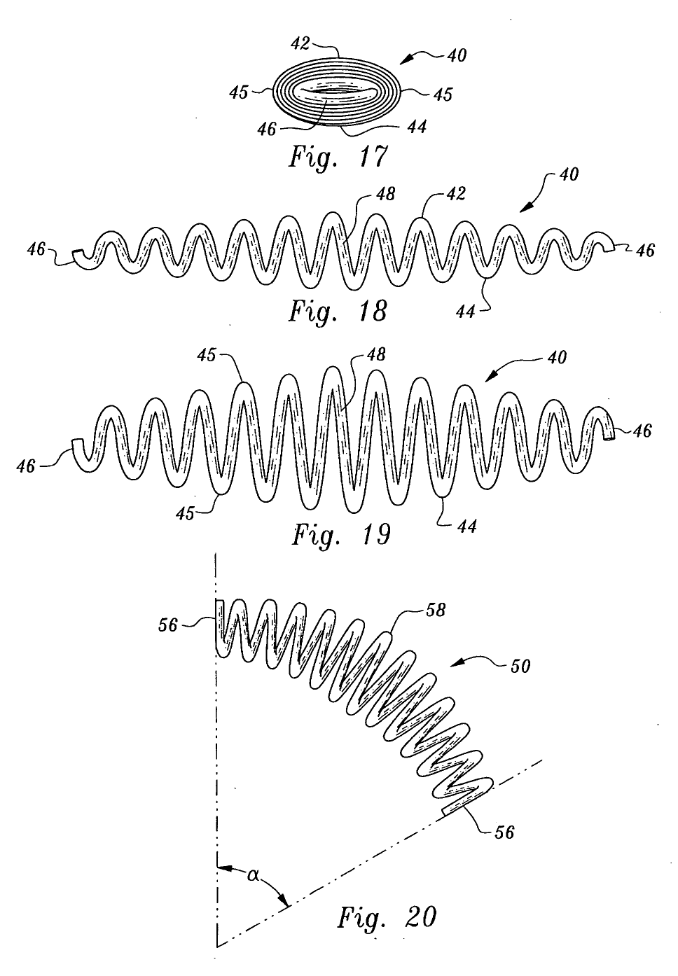

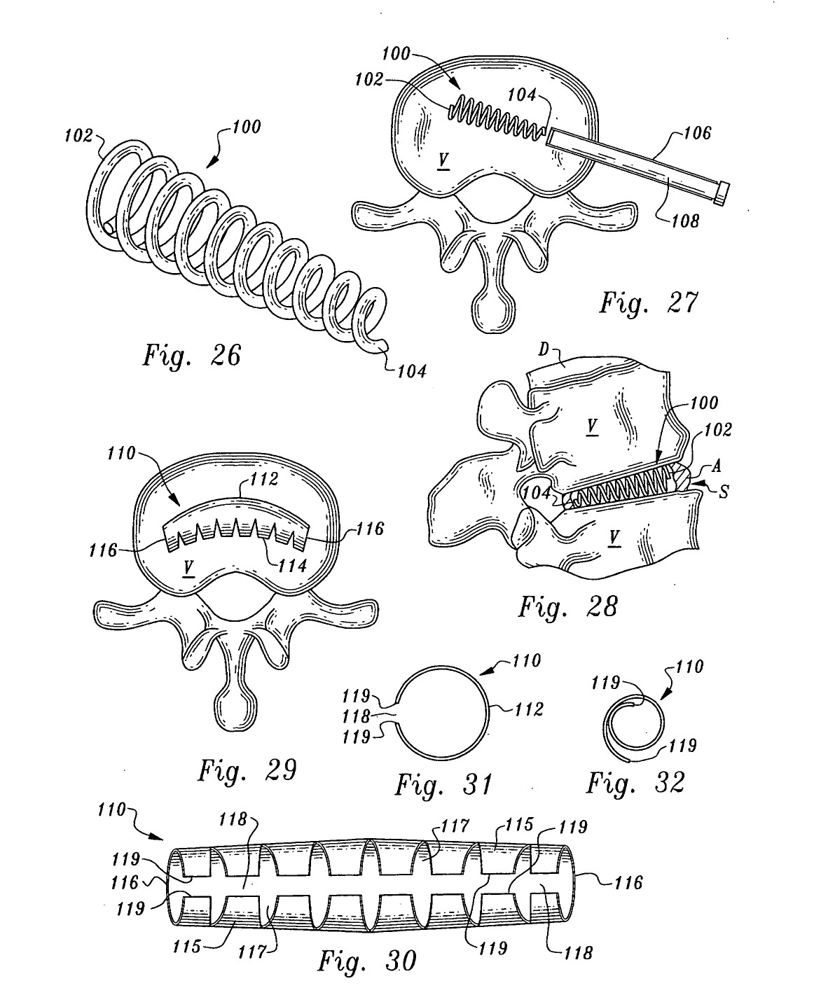

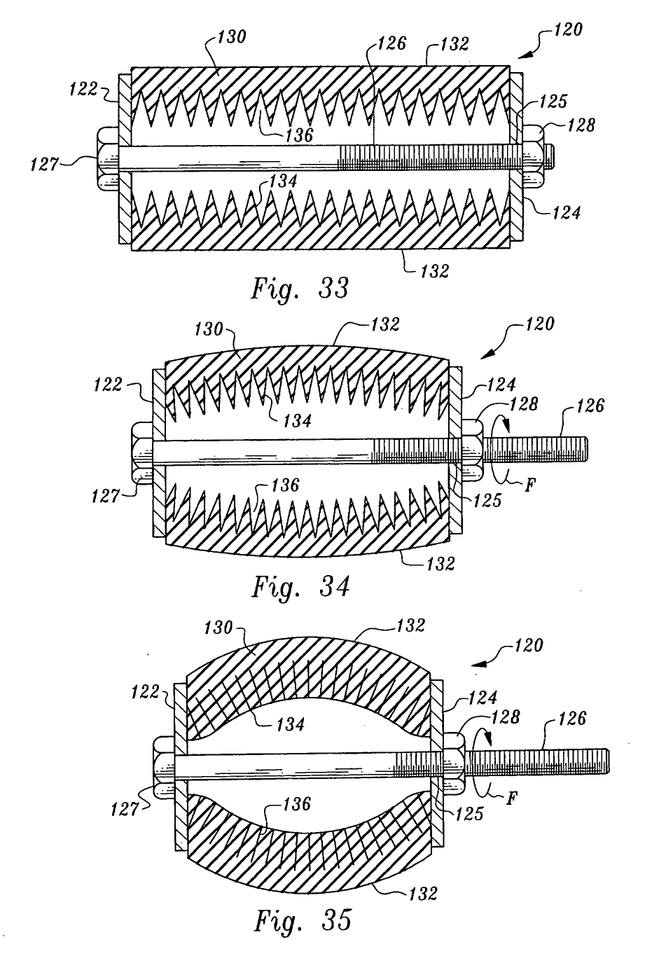

Referring to the drawings, wherein like reference numerals represent like parts throughout the various drawing figures, reference numeral 10 is directed to a toroid spring (FIG. 4) which provides a first embodiment of an implant according to this invention. The implant 10 of this and the other embodiments is adapted for placement within an intervertebral space S between adjacent vertebrae V, replacing at least a portion of a nucleus of a disk D. The implant can take the form of other embodiments including a barrel spring 30 (FIG. 9), an ellipsoid spring 40 (FIGS. 17-19), an arcuate spring 50 (FIG. 20), a cylindrical spring 60 (FIG. 21), a conical spring 100 (FIG. 26), a shell spring 110 (FIG. 30) or a tension spring 120 (FIGS. 33-35), as well as various combinations of these embodiments or other embodiments within the spirit of this disclosure.

In essence, the implant is preferably in the form of a helical spring having multiple turns. The helical spring can exhibit a constant diameter for each turn or have varying diameters for each turn, as well as other geometric modifications depending on the geometry desired for the implant.

The helical spring includes turns of material extending around a center line. The material does not typically intersect this center line. Rather, the center line defines a line generally following center points of each turn of the helical spring. This center line can be linear or curved. If curved, the curve can form an entire circle or merely a portion of an arc.

This center line is preferably oriented within a single plane and that plane preferably is oriented between adjacent vertebrae when the implant is in place within the intervertebral space S. Thus, the helical spring experiences compression loads provided by compression of the vertebrae V toward each other which are generally perpendicular to the center line. The turns of the helical spring are hence not brought toward each other or away from each other during loading, but rather the turns experience a distorting load tending to compress a height of each of the turns when the compression loads are encountered. Further details of the implant in general are illustrated by each of the embodiments described in detail below.

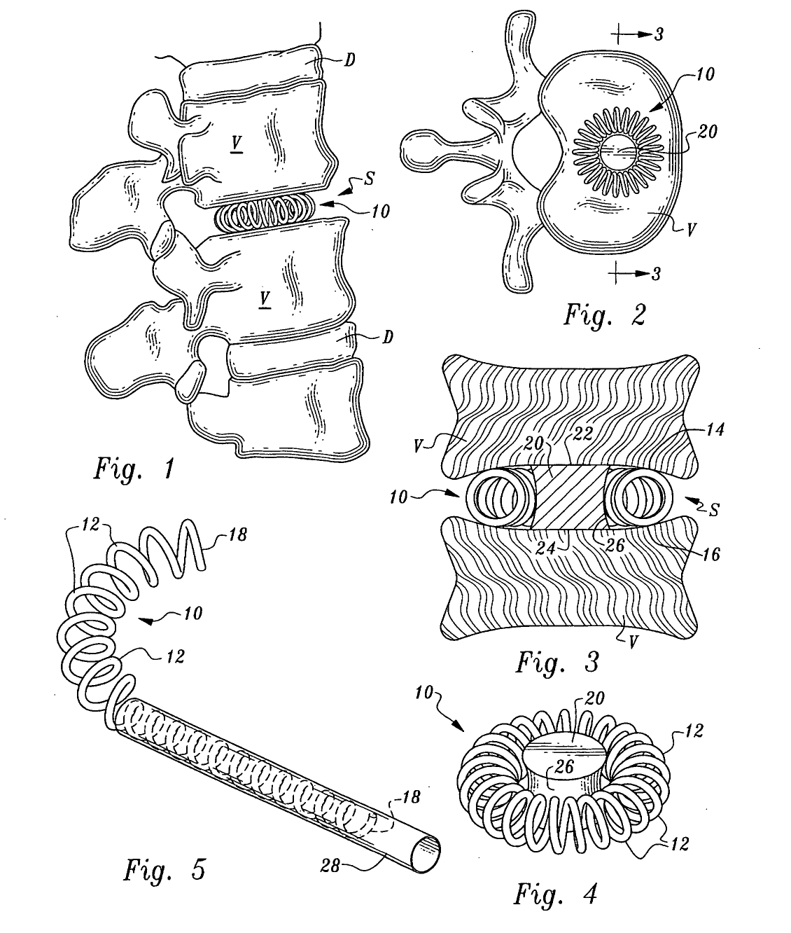

With particular reference to FIGS. 1-4, details of the toroid spring 10, providing a first embodiment for the implant of this invention, are described in detail. The toroid spring 10 preferably has a general outline in the form of a toroid (i.e. a donut) with multiple turns of the helical spring wrapping around a generally circular center line. Each of the turns 12 include an upper surface 14 opposite a lower surface 16. The upper surface 14 and lower surface 16 are compressed toward each other when vertical loads are experienced by the vertebrae V adjacent the toroid spring 10. While the toroid spring 10 could be continuous, most preferably it has ends 18.

Optionally, the toroid spring 10 includes a grommet 20 placed within a center of the toroid spring 10. The grommet 20 includes a top 22 opposite a bottom 24 in a concave sidewall 26 circumscribing sides of the grommet 20. The grommet 20 desirably counteracts the centripetal force generated during axial loading.

As with other embodiments, the toroid spring 10 is designed to be implanted posterior-laterally in a minimally invasive or open procedure. Most preferably, the implant is formed of a nickel titanium alloy having shape memory and super-elastic properties, such as “nitinol” or similar material. Particularly, the toroid spring 10 is compressed, such as when in its softer martensite phase, to a smaller diameter for placement within the delivery cannula 28.

Such compression can either occur by decreasing a diameter of each of the turns 12 of the toroid spring 10 so that a greater number of turns 12 are provided which are each smaller in diameter, or the toroid spring 10 can be somewhat unraveled between its ends 18 so that it exhibits a fewer number of turns but is elongated. Such a form would typically be achieved by first cooling the toroid spring 10 into its martensite phase, and then stretching the toroid spring 10 between its ends 18 until it is approaching linear. It could then be fed into the delivery cannula 28. When the toroid spring 10 is advanced out of the cannula 28, it would, utilizing its shape memory properties, return to its original austenite form and take the curving shape and turn diameter desired within the intervertebral space S.

While the toroid spring 10 and other implants of this invention are preferably formed from nickel titanium alloys having the characteristics identified above, it is also possible that the toroid spring 10 or other implant could merely be compressed in an amount less than that exceeding the elastic limit of the material, without requiring any phase change between harder and softer phases of the material forming the toroid spring 10 or other implant. Thus, other biocompatible materials could be utilized. By remaining below the elastic limit of the material, the material can still function effectively as a spring once implantation within the space S is completed.

With particular reference to FIGS. 2-16, details of the barrel spring 30 of the second embodiment are described. The barrel spring 30 is formed of a helical spring which follows a generally linear center line. The barrel spring 30 includes a top 32 opposite a bottom 34 on each of the turns thereof. The top 32 and bottom 34 are preferably adjacent the vertebra V adjacent the space S into which the barrel spring 30 is to be implanted. Alternatively, the top 32 and bottom 34 can abut other intermediate structures which in turn are supported by the associated vertebra V. The barrel spring 30 further includes ends 36 opposite each other with a middle 38 between the two ends 36.

Preferably, the middle 38 has a diameter greater than that at the ends 36. This difference can be selected to match a contour of the vertebra V (FIG. 8) to maximize support provided between the vertebra V and the barrel spring 30. Alternatively, the middle 38 can be further enlarged so that the middle 38 is compressed when the vertebra V come together more than portions of the barrel spring 30 adjacent the ends 36 thereof. In this way, turns in the barrel spring 30 adjacent the middle 38 are the first to become distorted. When a particularly high level of compressive force is applied between the vertebra V, successively greater numbers of turns of the barrel spring 30 extending away from the middle 38 would become involved in supporting this compression load. With such an arrangement, the barrel spring 30 would function somewhat akin to that of a “leaf spring” in that it would provide a variable amount of spring force based on the amount of compression load provided.

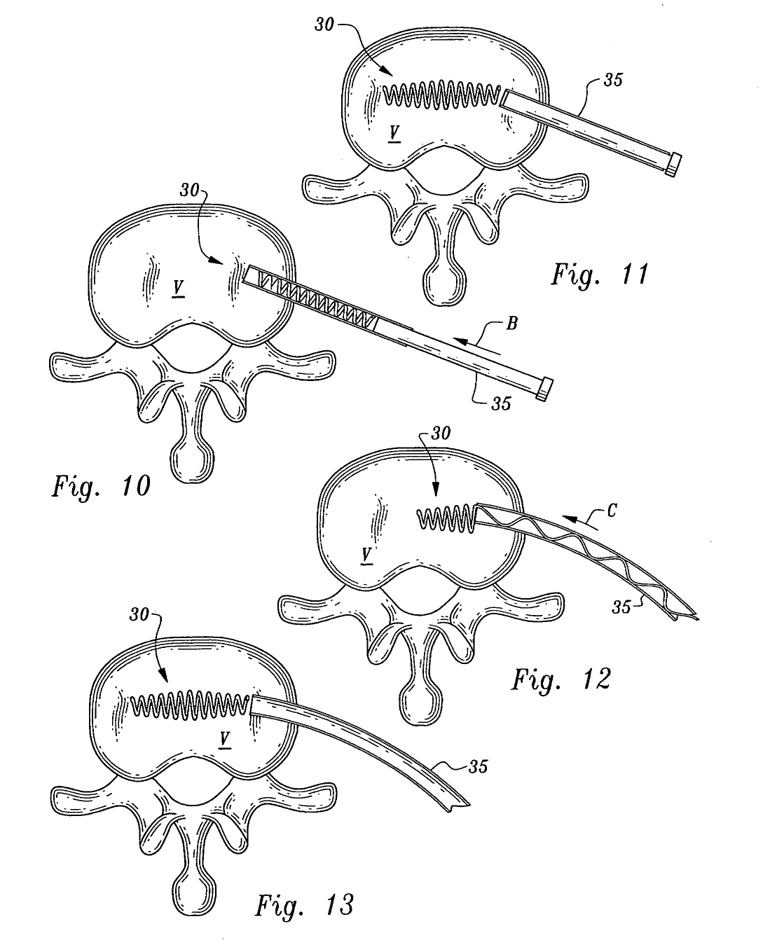

FIGS. 11-13 illustrate particular delivery cannulas 35 and the method for delivering an implant such as the barrel spring 30 into the space S (FIG. 8) between the vertebra V. As shown in FIG. 10, the barrel spring 30 begins within the delivery cannula 35. When a pusher is pushed (along arrow B of FIG. 10) the barrel spring 30 is caused to be discharged where desired. The barrel spring 30 or other implant would also typically become enlarged after being released from the delivery cannula 35. In FIGS. 12 and 13 the delivery cannula is shown curved in an arrangement which may be desirable depending on the incision site desired for advancing the barrel spring 30 or other implant to the space S between the vertebra V. As shown in FIG. 12, as the barrel spring 30 is advanced along the delivery cannula 35, it can be stretched out and then returned to its desired shape as it is released out of the end of the delivery cannula 35.

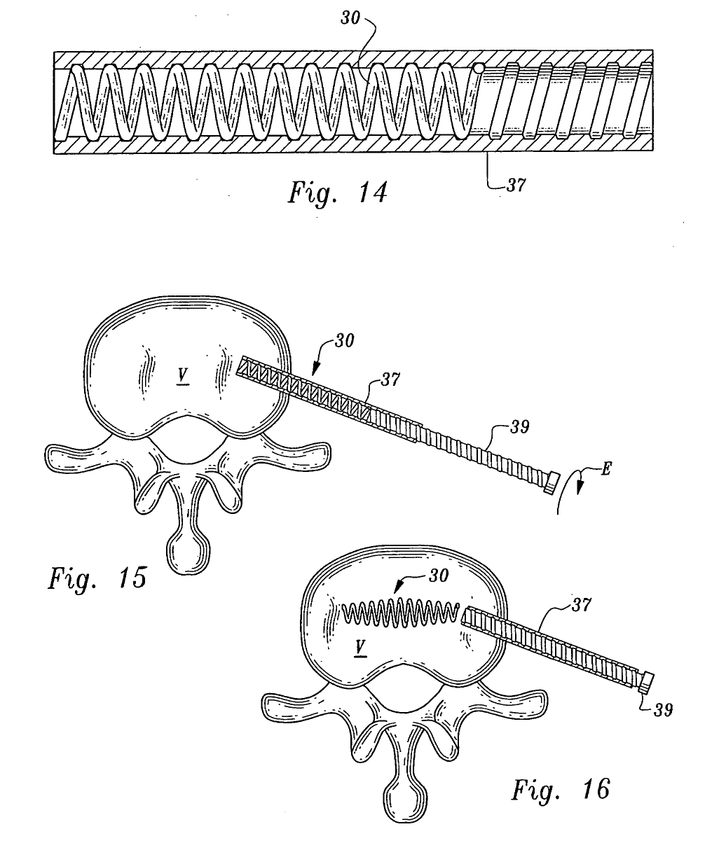

With particular reference to FIGS. 14-16, a variation on the delivery cannula 35 is provided in the form of a threaded cannula 37. The threaded cannula 37 includes threads on an inside surface thereof which approximately match a pitch of the turns of the barrel spring 30. A plunger 39 is provided which is threaded and can pass within the threaded cannula 37. As the plunger 39 is rotated (along arrow E of FIG. 15), it travels within the threaded cannula 37 and advances the barrel spring 30 in a rotating fashion (akin to that of a corkscrew) into the space between the vertebra V. As with other embodiments, the barrel spring 30 or other implant would preferably expand in diameter after being released from the threaded cannula 37. The pitch of threads in the cannula 37 can be altered to facilitate the desired turn pitch for the implant when compressed within the cannula, distinct from the turn pitch of the implant after expansion into the delivery site.

With particular reference to FIGS. 17-19, details of the ellipsoid spring 40 of the third embodiment are described. The ellipsoid spring 40 is similar to the barrel spring 30 except that it has an ellipsoid outline instead of a “barrel-like” outline. Particularly, the ellipsoid spring 40 includes a top 42 spaced from a bottom 44 by a height which is less than a width between opposite sides 45. Also, ends 46 opposite each other have turns of a lesser height than a height of turns adjacent the middle 48 of the ellipsoid spring 40. Thus, the ellipsoid outline of the ellipsoid spring 40 has a length between the ends 46 which is greatest, a height between the top 42 and bottom 44 which is least, and a width between the sides 45 which is intermediate between the height and the length. Other features of the ellipsoid spring 40 would be typically similar to those described above with respect to the barrel spring 30 or the toroid spring 10, or other embodiments disclosed below or within the scope of this disclosure.

With particular reference to FIG. 20, details of the arcuate spring 50, providing the fourth embodiment of the implant of this invention are described. The arcuate spring 50 is preferably similar in cross-section to that of the barrel spring 30. It could alternatively have a cross-section similar to that of the ellipsoid spring 40. Uniquely, the arcuate spring 50 follows a center line which curves. Most preferably, a curve of 60° defines the angle a shown in FIG. 20. The arcuate spring 50 extends between ends 56 and has a middle 58 therebetween which preferably is of greater height than a height of the arcuate spring 50 adjacent the ends 56. The arcuate spring 50 is particularly desirable in that it tends to match a contour of the vertebra V adjacent the space S (FIG. 3), particularly when the arcuate spring 50 has a cross-section which is ellipsoidal, such as that depicted in FIG. 17. Other details of the arcuate spring 50 are preferably similar to those discussed in other embodiments herein.

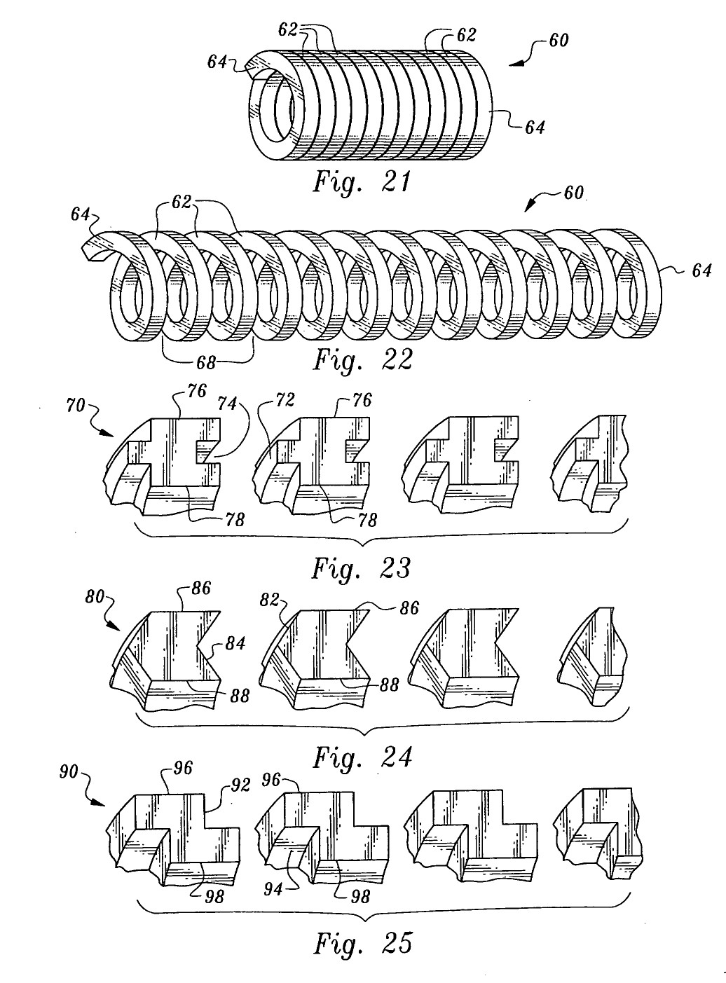

With particular reference to FIGS. 21-25, details of the cylindrical spring 60, providing a fifth embodiment of the implant of this invention, are described. The cylindrical spring 60 includes multiple turns 62 extending between ends 64. Uniquely, the cylindrical spring 60 has the turns 62 directly adjacent each other so that no significant gaps exist. The cylindrical spring 60 can still be stretched so that gaps 68 appear between the adjacent turns 62 (FIG. 22).

Most preferably, the cylindrical spring 60 has the turns 62 sufficiently close together so that the turns 62 can at least partially lock together or otherwise support each other. For instance, FIG. 23 depicts a first alternative turn pattern 70 where each turn 62 includes a tongue 72 opposite a groove 74 on sides of the turns between the outside 76 and the inside 78. The tongue 72 of one turn can rest within the groove 74 of an adjacent turn so that the turns 62 support each other. Such support is particularly desirable where a concern exists that the cylindrical spring 60 would be inclined to flatten not in a vertical fashion but in a somewhat diagonal fashion through sheer-like forces that would tend to collapse the cylindrical spring 60 in a somewhat sideways fashion.

The turns 62 are shown with a generally square cross-section between the substantially parallel outside 76 and inside 78. This cross-section could alternatively be circular (see FIG. 14 at the end of the implant) with or without structures to lock the adjacent turns 62 together. Also, the cross-section of each turn 62 could alternatively have other shapes such as rectangular with sharp or rounded corners, or elliptical. The turn 62 cross-section could also have an irregular shape. For instance, the outside 76 could be flatter than the inside 78, with the inside rounded.

FIG. 24 depicts a second alternative turn pattern 80 which features crests 82 opposite troughs 84 on sides of the turns 62 between the outside 86 and inside the 88. FIG. 25 depicts a third alternative turn pattern 90 which includes outside notches 92 complementally formed to mate with inside notches 94 on sides of each turn 62 between the outside 96 and the inside 98. With each of these turn patterns 70, 80, 90, some degree of support is provided between adjacent turns 62 of the cylindrical spring 60.

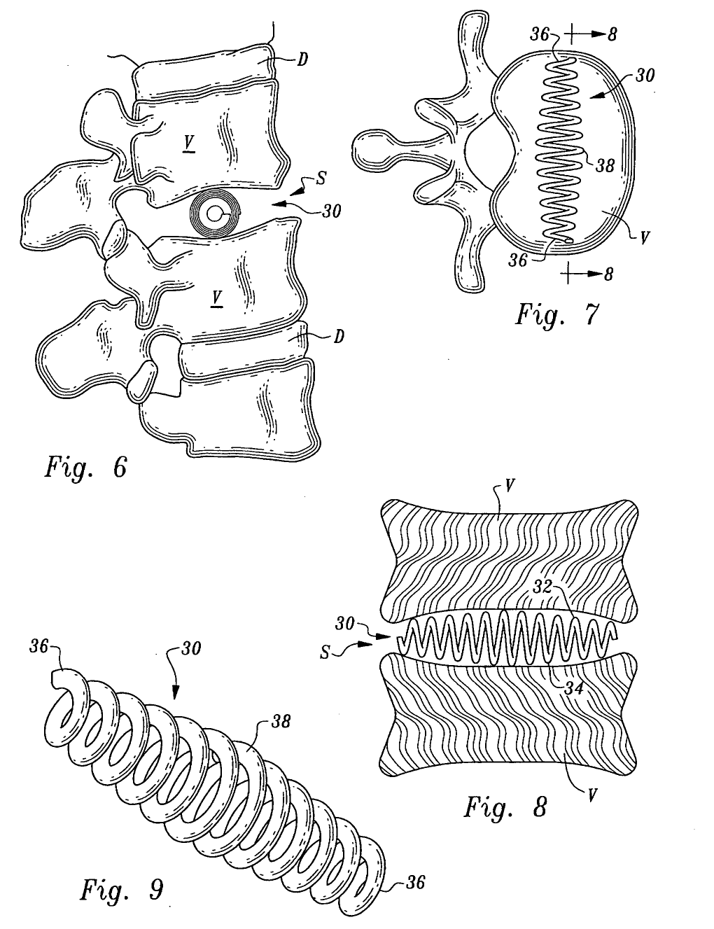

With particular reference to FIGS. 26-28, details of the frusto-conical spring 100, providing a sixth embodiment of this invention, are described. The frusto-conical spring 100 is generally similar to the barrel spring 30 of FIGS. 6-16 except that it has a generally frusto-conical outline. Particularly, a front end 102 has greatest diameter turns adjacent thereto and the rear end 104 has least diameter turns adjacent thereto.

A delivery cannula 106 can be provided which utilizes a rod 108 to advance the frusto-conical spring 100 into the space between adjacent vertebra V, in a manner similar to that discussed above with other embodiments. Uniquely, and as depicted in FIG. 28, the frusto-conical spring 100 has the ability to have the front 102 with the greater width provide for a certain amount of lardosis between adjacent vertebra V. It is often desirable to maximize a spacing between the vertebra V on an anterior side of the vertebra V. The conical spring 100 with its geometric configuration can provide for such lardosis. The annulus A of the disk D is also depicted in cross-section in FIG. 28. This FIG. 28 illustrates how the conical spring 100 acts as a nuclear replacement but does not replace the entire disk D. Rather, the annulus A preferably remains in place. This feature shown in FIG. 28 would preferably be similarly utilized in each of the embodiments of this invention.

With particular reference to FIGS. 29-32, particular details of a shell spring 110, providing a seventh embodiment of this invention are described. The shell spring 110 uniquely is not in the form of a helical spring. Rather, it has a generally “C-shaped” cross-section (FIG. 31) and acts somewhat like a complete cylinder but formed from a material with sufficient flexibility so that it can still provide the resiliency needed within the nucleus of the disk. The shell spring 110 includes an anterior side 112 opposite a posterior side 114. Preferably, the posterior side 114 includes a slit 118 therein extending between the ends 116 of the shell spring 110. Preferably, teeth 115 extend down to the slit 118 and up to the slit 118. Gaps 117 are provided between the teeth 115. The teeth 115 extend down to tips 119 defining extreme edges of the teeth 115 directly adjacent the slit 118.

The shell spring 110 functions in a manner similar to that of the other embodiments in that it is loaded vertically and has resiliency to allow it to flex somewhat and function as an at least partial replacement for the nucleus of the disk. To collapse the shell spring 110, it preferably has some of the teeth 115 overlapping the other teeth 115 so that the tips 119 rotate past each other (FIG. 32). When the shell spring 110 is formed from appropriate materials, such as nickel titanium alloys, it will readily expand to its original shape memory form when released from the delivery cannula. While the collapsed shell spring 110 is shown somewhat rolled up as in FIG. 32, alternatively, the teeth 115 could be offset from each other and the shell spring 110 could be collapsed so that the teeth 115 would be caused to go into gaps 117 on an opposite side of the slit 118, and with or without overlap. Preferably, the shell spring 110 has a certain amount of curvature, somewhat akin to the arcuate spring 50 of the fourth embodiment (FIG. 20).

With particular reference to FIGS. 33-35, a tension spring 120 is described, providing an eighth embodiment for the implant of this invention. The tension spring 120 preferably includes a first end plate 122 opposite a second end plate 124. A hole 125 is provided in the second end plate 124. Hence, a threaded shaft 126 can extend through the hole 125 and the threaded shaft 126 can also be coupled to the first end plate 122, such as through a head 127 attached to the threaded shaft 126. A nut 128 is provided which can advance along the threaded shaft 126 adjacent the second end plate 124. As can be seen, when the nut 128 is rotated about arrow F (FIGS. 34 and 35) the second end plate 124 is caused to be drawn toward the first end plate 122.

An expansion cylinder 130 is interposed between the end plates 122, 124. The expansion cylinder 130 is preferably formed from a resilient material, such as a hydrocarbon material that is biocompatible and has sufficient strength and resiliency characteristics. When the expansion cylinder 130 is compressed between the end plates 122, 124 an outside surface 132 thereof is caused to bulge outwardly. The expansion cylinder 130 thus takes on a somewhat barrel-like outline, similar to that of the barrel spring 30. An inside surface 134 is preferably provided with grooves 136 to facilitate such bulging. When the expansion cylinder 130 has completely bulged, the grooves 136 have been collapsed and the expansion cylinder 130 thus has a maximum resilient strength configuration. Excess portions of the threaded shaft 126 can be removed once the end plates are positioned where desired.

Each of the embodiments identified above are provided to illustrate the numerous different ways that implants can be provided according to this invention to provide for resilient disk nucleus replacement or augmentation, preferably within the annulus, but alternatively in place of both the nucleus and the annulus. With each of the embodiments of the implant, it is typically most desirable that the vertebra V not be fused together, but that the disk D continue to function as nearly to the disk's original function as possible.

Alternatively, when fusion of the vertebra is deemed necessary, the implants could alternatively be utilized along with the annulus (or not) to support the vertebra V during bone in-growth. Particularly, the implant might be configured to maximize a spacing between the vertebra V and the appropriate preparation of surfaces of the vertebra V would take place. Also, a bone growth media would be typically introduced to encourage bone growth into the region between the adjacent vertebra V.

In at least one scenario, a patient complaining of back pain can initially have an implant such as one of the embodiments identified above surgically implanted, preferably in a minimally invasive fashion, to replace the damaged nucleus of the disk. If this procedure results in cessation or satisfactory reduction in pain and other negative conditions, no further procedures would be necessary. However, if an undesirably high level of pain persists such that fusion of the adjacent vertebra V is considered to be warranted, the same implant already in place could conceivably be utilized, either with or without additional stabilization, and an additional procedure could be performed to prepare the vertebra V and introduce bone growth media to complete the fusion procedure.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1. An implant for location within an intervertebral space between a pair of adjacent vertebrae, the implant comprising:
a helical spring having a plurality of turns about a center line;
the helical spring adapted to be located with said center line between the two vertebrae; and
at least one of said turns adapted to have a turn height of at least half of a height of the space between the two vertebrae.

2. The implant of claim 1 wherein said center line lies within a center line plane, said center line plane adapted to pass between the two vertebrae when said helical spring is located between the two vertebrae.

3. The implant of claim 2 wherein said center line is substantially linear.

4. The implant of claim 2 wherein said center line is curving.

5. The implant of claim 4 wherein said center line forms a circuit.

6. The implant of claim 5 wherein said center line is circular.

7. The implant of claim 1 wherein said turn height of said at least one turn is substantially similar to a height of the space between the two vertebrae.

8. The implant of claim 1 wherein said helical spring exhibits a substantially toroidal outline.

9. The implant of claim 1 wherein said helical spring exhibits a substantially cylindrical outline.

10. The implant of claim 1 wherein said helical spring exhibits a substantially barrel shaped outline with ends of said helical spring shorter in height than a middle portion of said helical spring.

11. The implant of claim 1 wherein said helical spring is substantially ellipsoidal in outline.

12. The implant of claim 11 wherein said helical spring is shorter than it is wide.

13. The implant of claim 1 wherein said helical spring is substantially frusto-conical in outline with a front end having a height greater than a height of a rear end of said helical spring.

14. The implant of claim 1 wherein said helical spring is formed of a nickel titanium alloy having a martensite phase and an austenite phase, said spring adapted to be elongated along said center line and decreased in diameter away from said center line, and placed within a delivery cannula having a diameter less than said turn height after discharge from the cannula and transition of said helical spring from said martensite phase to said austenite phase.

15. The implant of claim 1 wherein said turns adjacent a middle of said spring have a height greater than turns of said spring adjacent ends of said helical spring.

16. The implant of claim 1 wherein said turns adjacent a front end of said helical spring have a height greater than a height of turns adjacent a rear end of said helical spring.

17. The implant of claim 1 wherein said turns have said turn height less than a turn width, such that a cross-sectional outline of said helical spring is somewhat elliptical.

18. The implant of claim 1 wherein said turns are located abutting each other when said helical spring is at rest.

19. The implant of claim 18 wherein said turns include complemental surfaces to provide some degree of locking when said complemental surfaces abut each other.

20. The implant of claim 19 wherein at least one of said turns includes a tongue extending therefrom and at least one of said turns includes a groove thereon sized to receive said tongue therein.

21. The implant of claim 19 wherein at least one of said turns includes a trough extending therefrom and at least one of said turns includes a crest thereon sized to reside within said trough of an adjacent said turn.

22. The implant of claim 19 wherein at least two of said turns abutting each other include complementally formed mating notches therein.

23. A method for delivery of an intervertebral space implant, including the steps of:
removing at least a portion of a nucleus of a disk within the intervertebral space;
locating a delivery cannula with a delivery end adjacent the intervertebral space;
providing an implant within the cannula, the implant including a helical spring having a plurality of turns about a center line, the helical spring adapted to be located with the center line between the two vertebrae; and
advancing the implant out of the cannula and into the intervertebral space with the center line of the implant between the two vertebrae.

24. The method of claim 23 including the further steps of compressing the implant from a larger at rest size to a smaller compressed size, locating the compressed implant within the cannula, and later expanding the implant when the implant is advanced out of the cannula and into the intervertebral space.

25. The method of claim 24 wherein said compressing step includes the step of forming the implant from a nickel titanium material having a softer martensite phase and a harder austenite phase and cooling the implant sufficiently to transition the implant into its martensite phase before compressing the implant according to said compressing step.

26. The method of claim 24 wherein said compressing step includes the step of elongating the implant.

27. The method of claim 23 wherein said providing step includes the step of sizing the implant to have a turn height for at least one of said turns which is at least half of a height of the intervertebral space.

28. The method of claim 27 wherein said sizing step includes sizing at least one of the turns to have a turn height substantially similar to a height of said intervertebral space.

29. The method of claim 23 wherein said providing step includes shaping the helical spring to exhibit a substantially toroidal outline.

30. The method of claim 23 wherein said providing step includes the step of shaping the helical spring to exhibit a substantially barrel shaped outline with ends shorter than a middle thereof.

31. The method of claim 23 wherein said providing step includes the step of shaping the helical spring to exhibit a substantially ellipsoidal outline.

32. The method of claim 31 wherein said shaping step includes the step of shaping the helical spring to be shorter than it is wide.

33. The method of claim 23 wherein said providing step includes the step of shaping the helical spring to be substantially frusto-conical in outline with a front end having a height greater than a height of a rear end.

34. The method of claim 23 wherein said providing step includes the step of shaping the helical spring to have turns adjacent a middle of the helical spring having a height greater than a height of turns adjacent each end of the helical spring.

35. The method of claim 23 wherein said providing step includes the step of adapting at least two of the turns to be abutting each other and shaped to engage each other along abutting surfaces thereof.

36. The method of claim 23 wherein said advancing step includes the step of rotating the implant within the cannula to advance the implant out of the cannula and into the intervertebral space.

37. The method of claim 23 wherein said advancing step includes the step of sliding the implant out of the cannula and into the intervertebral space.

38. A method for delivery of an intervertebral space implant, including the steps of:
removing at least a portion of a nucleus of a disk within the intervertebral space;
locating a delivery cannula with a delivery end adjacent the intervertebral space;
providing an implant within the cannula, the implant having a compressed size at least as small as a size of the cannula and an expanded size greater than a size of the cannula;
advancing the implant out of the cannula and into the intervertebral space; and
transitioning the implant from its compressed size to its expanded size, the expanded size at least half of a height of the intervertebral space.

39. The method of claim 38 including the further step of configuring the implant as a slitted cylinder.

40. The method of claim 39 wherein said configuring step includes the step of overlapping tips of the implant adjacent opposite sides of a slit in the slitted cylinder when the implant is at its compressed size.

41. The method of claim 39 wherein said configuring step includes the step of forming the implant from a nickel titanium alloy having a softer martensite phase and a harder austenite phase with said implant transitioning from said softer martensite phase to said harder austenite phase during said advancing step.

42. The method of claim 38 including the further step of configuring the implant to include a helical spring with a plurality of turns and with said helical spring having the compressed size including the helical spring elongated between ends thereof.

43. The method of claim 38 including the further step of configuring the implant to include a pair of end plates with a shaft therebetween and with a cylinder of resilient material surrounding the shaft and abutting each of the second end plates, and located between the two end plates, the cylinder of resilient material adapted to exhibit radial expansion upon axial compression of the cylindrical resilient material when axially compressed by the end plates.

44. The method of claim 43 wherein said configuring step includes the cylinder formed of resilient material including a cylindrical outside surface and a generally cylindrical inside surface, the inside surface including a plurality of grooves thereon which become narrower as the cylinder of resilient material is compressed and radially expanded.

45. The method of claim 44 including the further step of cutting off portions of the shaft which are excess after the cylinder of resilient material has been compressed axially and expanded radially.
